(19) United States
(12) Patent Application Publication
(10) Pub. No.: US 2022/0273240 A1
(43) Pub. Date: Sep. 1, 2022

(54) NEEDLE SENSOR AND METHOD OF MANUFACTURING THE SAME

(71) Applicant: LAXMI THERAPEUTIC DEVICES, INC., Goleta, CA (US)

(72) Inventor: Matthew Hopcroft, Santa Barbara, CA (US)

(21) Appl. No.: 17/186,605

(22) Filed: Feb. 26, 2021

(51) Int. Cl. A61B 5/00

(52) U.S. Cl. CPC A61B 5/6848; A61B 5/0031; A61B 2560/0214

(57) ABSTRACT

A sensor for facilitating analysis of biological specimens in vivo includes a base having first and second ends, a rigid needle that extends away from the first end for piercing skin, the needle having a tip including at least a first conductive layer forming a first electrode, a second conductive layer forming a second electrode, and a first insulating layer between and electrically insulating the first and second conductive layers from one another, and at least first and second electrical contacts at or near the second end of the base. The first electrical contact is in electrical communication with the first electrode, the second electrical contact is in electrical communication with the second electrode, and the first and second electrical contacts are electrically insulated from one another.

### BACKGROUND

### Field

The present disclosure generally concerns medical devices and tools used for measuring analytes in the body. More specifically, the disclosure relates to a needle sensor of the medical device that may be implanted into or otherwise access the body via a small incision to facilitate the measuring of the analytes in the body.

### Description of Related Art

Recent years have seen increased interest in wearable sensors for measuring analytes in the body (e.g., glucose levels), because such sensors might greatly ease analyte measurement. In particular, there has been growing interest for wearable sensors that selectively measure analytes that must be continuously monitored or at least more frequently monitored. For example, monitoring glucose levels is particularly important for individuals suffering from type 1 or type 2 diabetes, as well as for example, individuals such as pregnant women who develop gestational diabetes, and simply to monitor individuals' general wellness, among other reasons. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications.

Traditionally, glucose has been monitored in capillary blood, typically from a fingertip. A user of these traditional glucose measuring devices would prick a fingertip with a blood lancet to produce a drop of blood. The user would place the drop of blood on a test strip and insert the test strip into a glucose meter, where the glucose meter would then measure the glucose concentration in the drop of blood. Although widely used, traditional glucose monitoring devices are limited because they can only measure blood glucose levels at a single point in time. This can be problematic because blood glucose levels can fluctuate throughout a day, especially after food is digested. Regular monitoring of blood glucose levels may require multiple finger pricks and multiple measurements. However, frequent blood pricks can be painful or otherwise overly intrusive, and users may be hesitant to take a sufficiently appropriate number of blood glucose measurements to get any sort of meaningful semi-continuous data. In addition, for example, inconsistent user and environmental factors based on minor variances with each separate finger prick may also cause errors and/or other inconsistencies in compiling such semi-continuous data.

### SUMMARY

In contrast to traditional glucose monitors, wearable devices can allow for continuous glucose monitoring (e.g., continuous glucose monitors (CGM)). CGMs are always active and continuously measure glucose levels at set intervals (e.g., measured every ten seconds and readings reported every five minutes). The continuous data can allow users to track glucose levels over the course of a day or over a few days, and help them make more informed decisions about their diet, physical activity, and medications. CGMs operate by inserting a small sensor under the skin, typically on the abdomen or on the back of the upper arm, but can also be inserted at any other suitable location on the human body, for example, on the lower arm. FIGS. 1A and 1B show examples of wearable devices that are worn on a human body, where in FIG. 1A a CGM 1 is worn or otherwise attached to an abdomen or torso region of a patient or subject, and in FIG. 1B the CGM 1 is alternatively worn or otherwise attached to an upper arm of a patient or subject. Other sensors may also be worn on other parts of the body. The sensors can be held in place by an adhesive and can wirelessly transmit glucose measurements to, for example, a separate monitoring device. However, unlike traditional glucose monitors, CGMs may instead measure glucose in interstitial fluid (e.g., glucose in the fluid between cells) rather than glucose in blood.

An example of a wearable CGM according to an embodiment of the invention is schematically shown in FIGS. 2A to 3. FIG. 2A shows a perspective view of an embodiment of a wearable CGM 1, FIG. 2B shows a side view of the wearable CGM 1, and FIG. 2C shows a bottom view of the wearable CGM 1. The CGM 1 may generally include a body or housing 10 having a top end 11 and a bottom end 12. An adhesive 13 may be found on the bottom end, and may be in the form of, for example, a tape that has a perimeter that extends slightly outside of the perimeter of the housing 10, to improve adhesion to the patient's body. At the bottom end 12, there may also be a hole or opening 14, through which a sensor extends. The sensor 15 can be integrated on a needle, to form a needle sensor 15. The needle sensor 15 may only protrude slightly out of the bottom end 12, for example, about 2 mm, and may be connected in the housing to other circuitry. FIG. 3 shows a schematic block diagram of the CGM 1. The needle sensor 15 may be connected to a PCB 16 or other circuitry. The PCB 16 may further include or be otherwise connected to a transmitter/receiver 17, a controller 18, and a battery 19. The transmitter/receiver 17 allows the CGM 1 to communicate with a separate monitoring device, the controller 18 can control functionality and monitoring of the CGM 1, and the battery 19 can supply power to the components of the CGM 1.

The above analyte measurement methods, including monitoring with CGMs, are still categorized as invasive, that is, they require entry into the body or a body cavity (e.g., where a probe is used to percutaneously access the body fluid being analyzed). In contrast, non-invasive glucose monitoring methods have also been explored, but have proven difficult to implement for various reasons such as reduced accuracy due, for example, to extremely low testing sample volumes, varying sweat rates, sample evaporation, and variances in correlation between blood and sweat glucose concentrations, among other factors. Meanwhile, invasive methods have traditionally used a needle to pierce the skin and to access the interior of patients and other biological specimens while causing minimal damage, in order to extract an external blood sample or, for example, to provide a small opening for introducing a separate soft flexible sensor. In contrast, there has historically, been limited or no interest or success in integrating analyte sensors directly onto needles intended to remain in the body because of recognized difficulties in designing and manufacturing a sufficiently compact and reliable needle-based system, and for example, for patient comfort reasons. To address this and similar issues, needles according to embodiments of the invention can, for example, be integrated with electrodes that can be used for various applications, including electrochemical and other types of sensing. Such needles can be made sufficiently small, so that, for example, patient discomfort during insertion and continuous wearing can be minimized, while the sensing properties of the CGMs remain robust.

An example of a needle sensor for a CGM according to one embodiment of the invention is schematically shown in FIGS. 4A-4C. FIG. 4A shows a perspective view of a needle sensor 15, FIG. 4B shows a top view of the needle sensor 15, and FIG. 4C shows a close-up view of a tip of the needle sensor 15 including the sensor portion. The needle sensor 15 includes a base 151, a shaft 152, and a needle tip 153. The needle tip 153 may include various electrodes 154a, 154b, and 154c that facilitate the glucose monitoring. The electrodes 154a, 154b, 154c are electrically connected to corresponding larger contacts 155a, 155b, 155c at the base, for easy connectivity with the PCB 16 (see FIG. 3).

Moreover, problems particularly arise with cost-effective manufacturing of small and structurally robust needles. Very small needles (e.g., microneedles) are desired to reduce sensor size and minimize user sensation (e.g. pain or discomfort) during needle insertion, but most methods of manufacturing microneedles produce needles of silicon (via conventional silicon micromachining or use of structural lithography resists, e.g., SU-8 photoresists), or plastic needles (via micromolding methods). Unfortunately, most silicon and plastic needles are not structurally robust, thus requiring larger dimensions, and/or are not biocompatible.

Embodiments of the present disclosure include a method of constructing a needle with integrated electrodes. The needle can be constructed from alternating layers of conductive material (e.g., electrodes) and insulating material that insulate the separate layers of conductive material from one another. Structures with this construction can be described as a laminate structure with a laminate-type construction, and the structure can be formed or cut in the shape of needle. This arrangement is particularly well suited to fabrication by multilayer deposition and patterning methods such as wafer-scale microtechnology and flexible electronic circuit manufacturing. In some embodiments, the needle and electrodes can be fabricated in a unified fabrication process, which may be faster and cheaper than other methods. It also allows a needle to be constructed with electrodes integrated into the mechanical structure of the needle, which increases robustness and reliability. In some embodiments, the shaft and/or other portions of the needle aside from the exposed tip can be insulated with a separate coating. In other embodiments, the insulating layers can be extended or otherwise patterned to integrally insulate certain portions of the needle.

### BRIEF DESCRIPTION OF THE DRAWINGS

Further features and advantages of the invention will become apparent from the description of embodiments using the accompanying drawings. In the drawings:

FIGS. 1A and 1B schematically show a wearable continuous glucose monitor (CGM) being worn on a patient's abdomen and on a patient's arm, respectively.

FIG. 2A-2C respectively show a perspective view, a side view, and a bottom view of an embodiment of a CGM according to an embodiment of the invention.

FIG. 3 shows a schematic block diagram of the CGM of FIG. 2.

FIGS. 4A-4C respectively show a perspective view, a top view, and an enlarged view of a tip of a needle sensor of the CGM of FIGS. 2A-3.

FIG. 5 shows a perspective view of a laminate structure used to manufacture a needle sensor of the CGM of FIGS. 2A-3.

FIG. 6A shows a needle tip formed from the laminate structure of FIG. 5.

FIG. 6B shows a needle tip formed from a laminate construction according to an alternative embodiment.

FIG. 7 shows a top view of an alternative needle sensor according to another embodiment of the invention.

FIGS. 8A-8C show various embodiments of a rear end of the needle sensor of FIG. 7, with various different electrical contact arrangements.

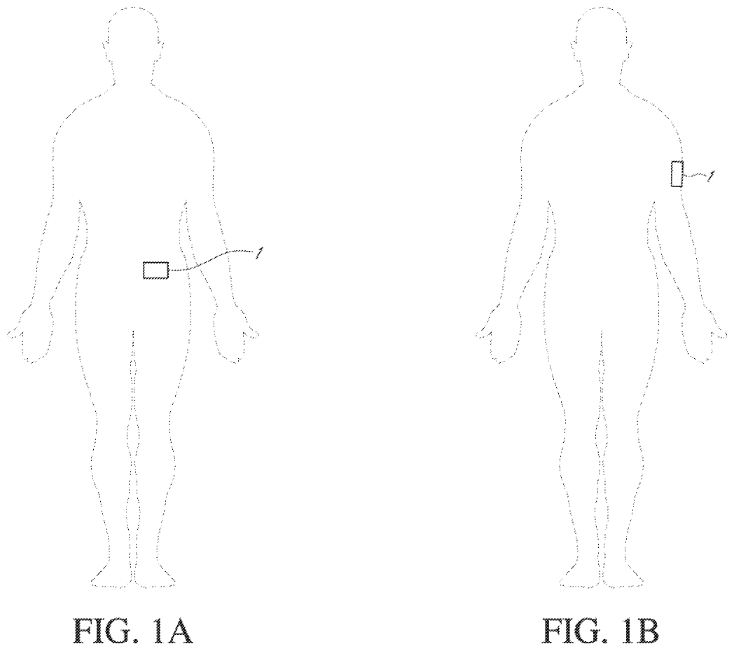

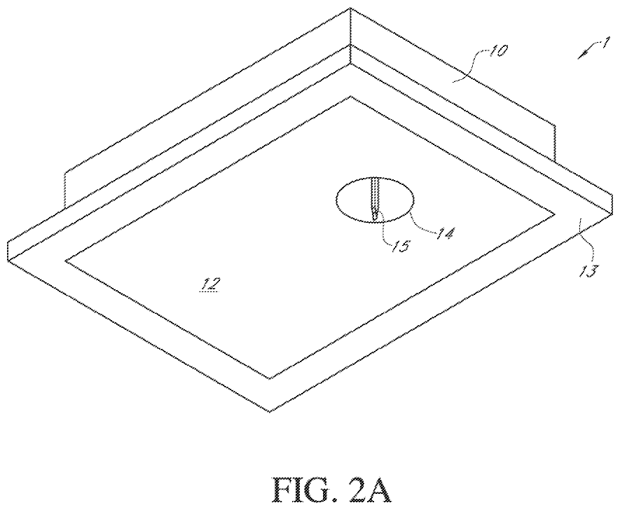

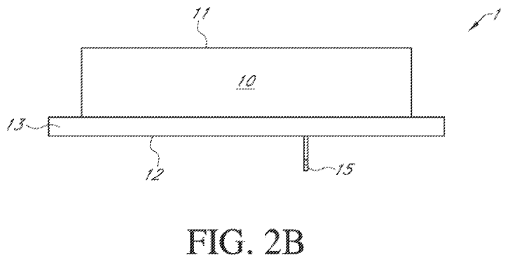

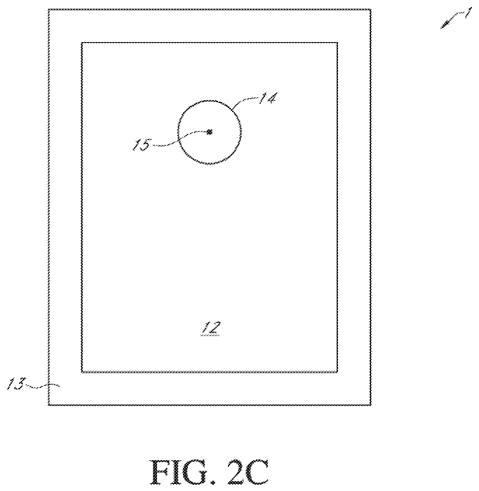

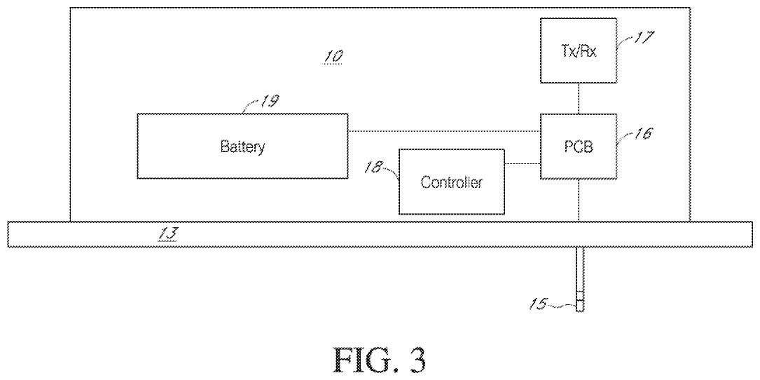

### DETAILED DESCRIPTION

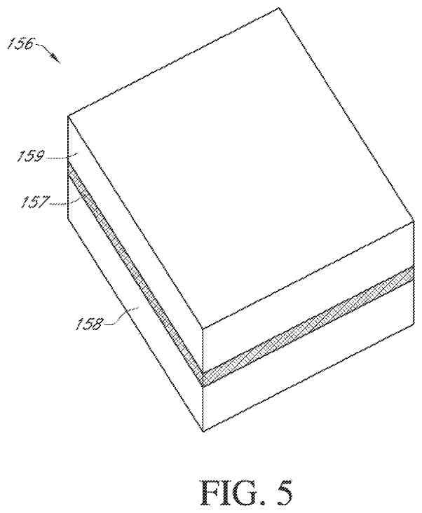

FIG. 5 schematically shows a perspective view of a laminate structure used to manufacture a needle sensor according to an embodiment of the invention.

A shown in FIG. 5, a laminate structure 156 can be formed by applying an insulating layer of material 157 on top of a first conductive layer of material 158. The first conductive layer 158 may serve in a more traditional sense as a substrate during the fabrication process, with the insulating layer 157 deposited or otherwise applied on the conductive layer 158. The insulating layer 157 may be thinner than the conductive layer 158, so long as it is thick enough to insulate the conductive layer 158. A second conductive layer of material 159 can then be applied on top of the insulating layer 157. The separate layers 157, 158, 159 can initially be formed in a larger wafer or sheet based format, and can be cut into smaller specific desired shapes thereafter, as discussed in greater detail below. Heat, pressure, adhesives, or other means may be applied as necessary to ensure that the laminate structure 156 behaves as a solid material.

In some embodiments, more than two electrodes may be required or desired. In such cases, as many conductive layers as needed, for example, three or more layers, may be formed, so long as insulating layers are also deposited between and separating the conductive layers from one another for the electrodes to function properly.

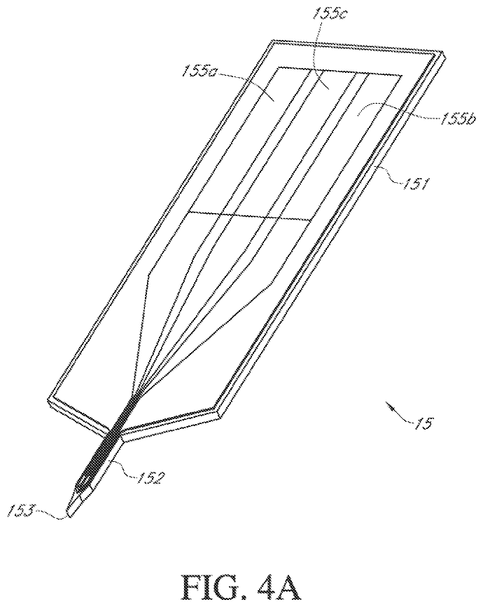

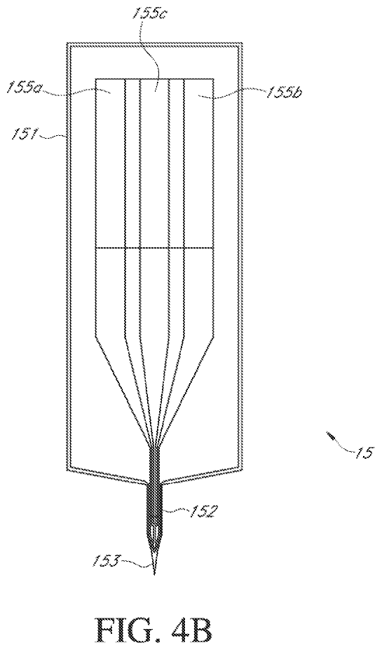

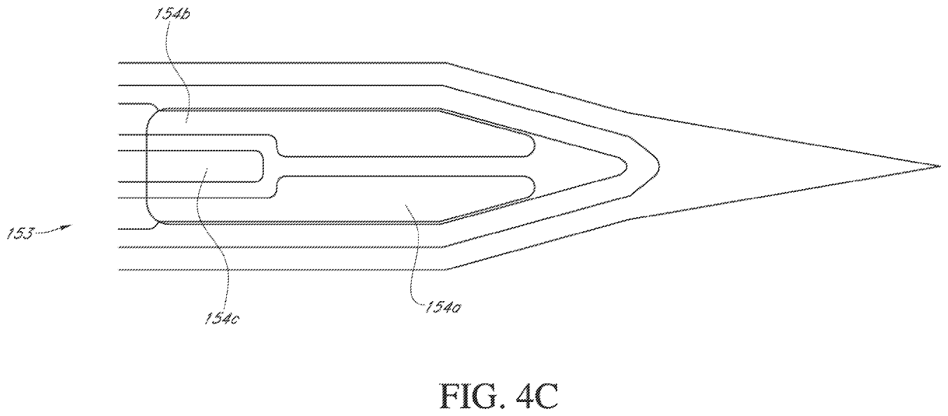

As noted above, after the layering process is completed, the laminate structure 156 can then be cut or otherwise formed into a needle shape with a sharp tip and a shaft connecting the tip to electrical contacts at or near a base or end opposite the tip. Refer, for example, to FIGS. 4A and 4B to show a general structure of a needle after it has been cut from a fabricated sheet or wafer. Depending on the size of the sheet or wafer formed and the desired size of each needle, a single fabricated wafer can potentially be used to form tens or even hundreds or more individual needle sensors. For embodiments of the needle sensor where an insulating layer is not previously formed to also insulate the entire needle, a separate insulating coating can then be applied to the needle shaft and base, where for example, the sensor tip and the electrical contacts remain exposed to communicate with external elements, an end user or other electrical components in the CGM.

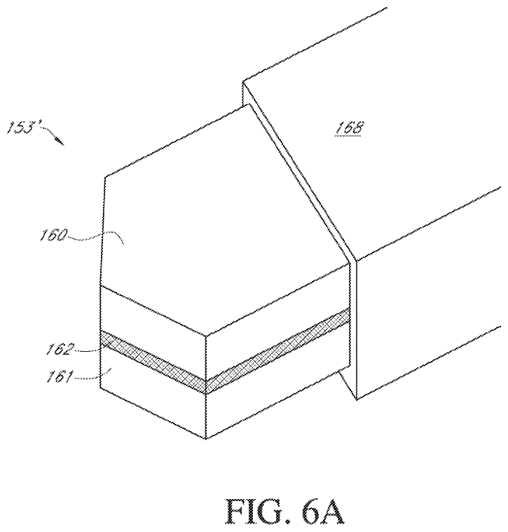

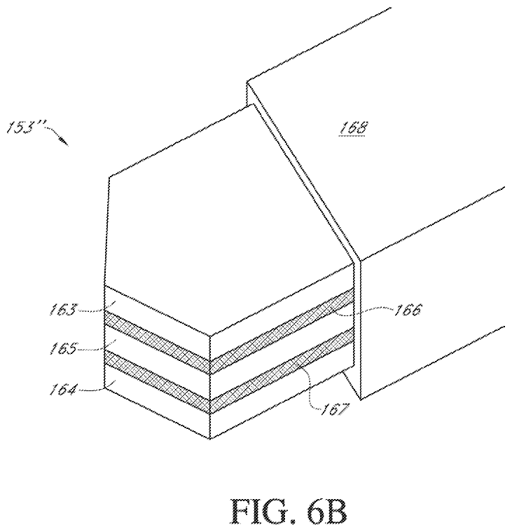

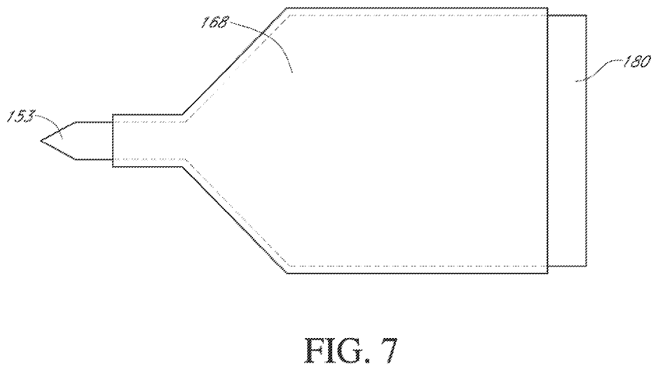

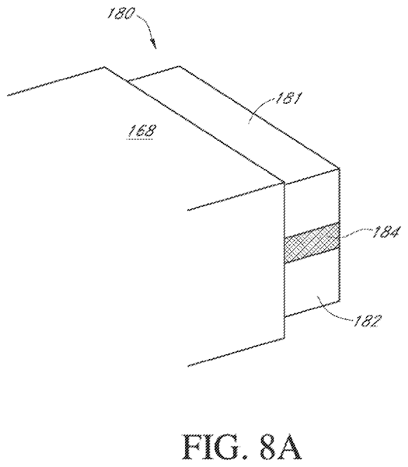

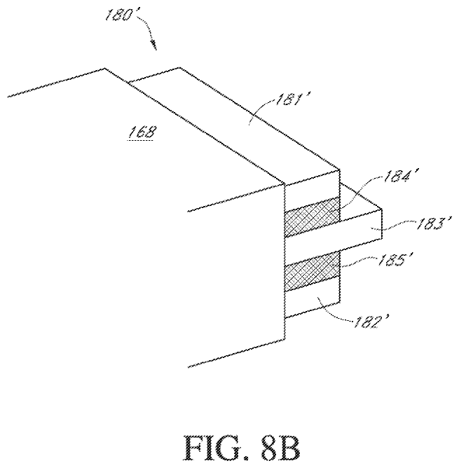

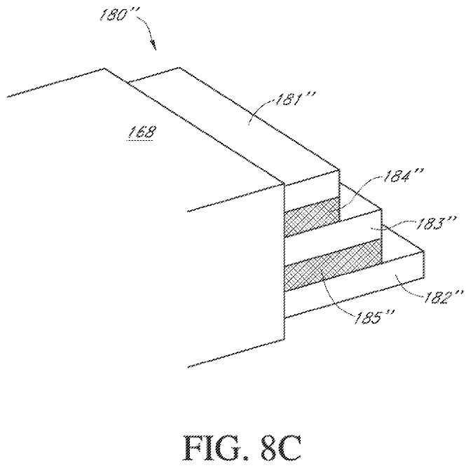

FIGS. 6A and 6B schematically show needle tips formed from a laminate structure such as laminate structure 156 discussed above with reference to FIG. 5. It is first noted that each of FIGS. 5, 6A, and 6B (as well as later FIGS. 7 to 8C) represent simplified schematic illustrations showing different structures of needle sensor embodiments, and that the figures are not drawn to scale. Furthermore, additional features and arrangements, for example, more complex layering schemes, may be employed without departing from the spirit and scope of the invention. FIG. 6A shows a needle tip 153′ with two electrodes according to one embodiment. In this embodiment, a first electrode 160 may be positioned at a top surface (e.g., an exposed surface) of the needle tip 153′. A second electrode 161 may be positioned at a bottom surface (e.g., an opposite exposed surface) of the needle tip 153′. The first electrode 160 may be, for example, a working electrode and the second electrode 161 may be, for example, a counter electrode, or vice versa. The electrodes may be made of the same or different materials, for example, a biocompatible metal, and the conductive layers may have the same or different thicknesses. The two electrodes are separated and insulated, for example, by an insulating layer of material 162. The insulating layer 162 may have a thickness that is less than a thickness of each of the electrodes 160, 161, but embodiments of the invention are not limited thereto, and in some alternative arrangements, a thickness of the insulating layer 162 may be the same as and/or greater than respective thicknesses of the electrode layers 160, 161.

FIG. 6B shows a needle tip 153″ with three electrodes according to a further embodiment. Other embodiments may include more than three electrodes. In the embodiment shown in FIG. 6B, a first electrode 163 may be positioned at a top surface (e.g., an exposed surface) of the needle tip 153. A second electrode 164 may be positioned at a bottom surface (e.g., opposite exposed surface) of the needle tip 153. A third electrode 165 may be positioned between the first and second electrodes 163, 164, and may for example, only be exposed along its perimeter extending around the needle tip. The first electrode 163 may be a working electrode, the second electrode 164 may be a counter electrode, and the third electrode 165 may be a reference electrode, although other arrangements may also be plausible in other embodiments. The material of the electrodes may be the same or different, and the conductive layers may have the same or different thicknesses. The third electrode 165 may be separated and insulated from the first electrode 163, for example, by a first insulating layer of material 166, and may be separated and insulated from the second electrode 164, for example, by a second insulating layer of material 167. The insulating layers 166, 167 may also have the same or different thicknesses from one another. In general, the insulating layers 166, 167 will have thicknesses that are less than a thickness of each of the electrodes 163, 164, 165, but embodiments of the invention are not limited thereto, and in some alternative arrangements, a thickness of each of the insulating layers 166, 167 may be the same as and/or greater than respective thicknesses of the electrode layers 163, 164, 165.

Additionally, outer portions and surfaces of the needle which are not exposed for electrical activity may be passivated or otherwise insulated in any of various different ways. For example, in some embodiments, the needles may first be cut or otherwise formed, and then may be insulated thereafter by applying a coating (e.g., a spray-coated polymer). FIGS. 6A, 6B, and 7 each schematically show an additional external insulation layer 168 surrounding portions of the needle structure that are not intended to be exposed for electrical activity, for example, so that the needle sensor tip and/or electrical contacts at an end of the needle sensor opposite the tip are the only portions not covered by the additional insulation layer. In FIGS. 6A and 6B, the outer insulating layer 168 leaves the electrodes at the tip of the needle sensor exposed, while for example covering the shaft of the needle to prevent undesired conductivity and/or other outside interference at the shaft region. Similarly, FIGS. 8A to 8C show different arrangements where insulating layer 168 leaves the electrical contacts at an opposite end of the needle sensor exposed. These are only select example arrangements, and in other embodiments, other parts of the needle sensor may also be exposed, or the tip and/or electrical contacts may be exposed in any of various different ways, based on the particular arrangements in each embodiment. In other embodiments, additional insulation may be achieved, for example, by increasing the extent or coverage of the fabrication of the insulating layers to cover the exposed edges of the conductive layers.

In other embodiments, the top surface, the bottom surface, or both may be formed of insulating material during layer fabrication (i.e., prior to cutting and forming of the individual needle sensors). For example, in some embodiments, a substrate or other initial deposit layer may instead be an insulating layer on which a first conducting layer is then applied. Furthermore, after the last conducting layer is formed, in some embodiments a further insulating layer may also be formed as a top layer, prior to the needle sensors being cut and formed. In such cases, one or more of the outer layers of the needle sensors may already be insulating immediately after cutting, and resulting in a similarly insulated final needle sensor product, so that further steps of adding an insulating coating and/or other passivation steps may not be needed.

In yet other embodiments, other external parts or features of the CGM may also provide insulation for the needle sensor. For example, in some embodiments, a separate needle sensor housing (not shown) can be provided to hold the needle sensor, and to surround and/or otherwise insulate at least some portions of the needle sensor.

As noted above, FIG. 7 schematically shows an entire needle sensor with an applied insulating coating 168 that functionally exposes the needle sensor tip 153 and electrical contacts 180 at an end of the needle sensor opposite the tip 153. The sensing area of tip 153 serves to introduce the electrically active sensing electrodes thereon into a biological tissue or other region of interest. The external insulating coating 168 minimizes or prevents unwanted conductivity or other interference from external factors at locations on the needle sensor other than the tip and the electrical contacts, to improve functionality and sensing accuracy. In addition, the signal from the sensing electrodes at tip 153 must be connected to electronic circuits for generating stimulus signals, recording electrical responses, and/or other capabilities. Some of these circuits may be located on the needle sensor, but others may be located away from the needle sensor and connected to the sensing electrode by external wires, for example, in other portions of the CGM. The electrical contacts 180 facilitate communication between the needle sensor and such external systems, for example, other circuitry in the CGM through external ports or other wire contacts, at a region of the needle sensor located away from the sensing region at the tip 153 so that they do not interfere with the insertion and/or sensing mechanisms, and may be formed in a variety of ways.

In one embodiment, the electrical contacts are all arranged on a single side or face of the needle sensor. Such an arrangement may make it easier to provide connectivity to the rest of the CGM (e.g., so that the CGM does not need to provide complementary docking contacts for both sides of the needle sensor). For example, in the arrangement shown above in FIGS. 4A to 4C, all of the contacts 155a, 155b, 155c are arranged on a same side of the needle sensor. In arrangements where the electrodes are layered as shown in the embodiments in FIGS. 6A and 6B, a similar contact arrangement can be achieved, for example, by arranging insulated vias during the fabrication process somewhere in the circuitry (not shown) between the needle sensor tip 153 and the electrical contacts 180. In this manner, for example, an electrode layer on one side of the needle sensor can be provided with electrical contacts that are arranged on an opposite side of the needle sensor, and any electrode layer that is sandwiched between separate outer conductive layers can similarly be provided with an electrical contact on the same side of the needle sensor.

In other embodiments, vias may not be necessary to provide for a more simplified and potentially more robust fabrication process. Instead, contact may simply be made to the exposed edges or other surfaces of the material provided for each electrode layer at an end of the needle sensor opposite the tip 153. In some embodiments, regions of the conductive materials may be exposed in a “stairstep” pattern by selective removal of material, as discussed in greater detail below.

FIG. 8A shows a first schematic arrangement of electrical contacts 180 provided at an end of the needle sensor opposite the tip 153. As can be seen in FIG. 8A, an insulating layer 168 exposes the entire rear end of the electrode layers to form the electrical contacts, but in other embodiments, only part of the rear end of the needle sensor may instead by exposed, so long as a region for each electrical contact is sufficiently exposed to provide a robust connection with a port or other external wiring for electrical communication with other components of the CGM.

In FIG. 8A, two separate conductive layers 181, 182 are provided, with an insulating layer 184 separating and insulating the conductive layers 181, 182 from one another. In this arrangement, the two conductive layers are simply exposed, with no additional removal or other shaping of the electrodes. Here, the exposed portions of conductive layer 181 provides for one electrical contact region on one side of the needle sensor, while the exposed portions of the conductive layer 182 provide for a second electrical contact region on an opposite side of the needle sensor. The side perimeters and/or end faces of the conductive layers 181, 182 may also be utilized for electrical communication with the other components in the CGM, depending on how the external port or contact wires are arranged.

In an alternative embodiment not shown, the arrangement in FIG. 8A can also be modified in a stepped manner, either by fabrication of differently sized layers or by selective removal of part of one conductive layer and the insulating layer, so that both electrodes are exposed to and can be accessed from a single side of the needle sensor, to facilitate potentially easier electrical connectivity with the other parts of the CGM. In yet other embodiments, only certain portions of one conductive layer can be removed instead of an entire end region, for example, one corner of one of the conductive layers, to similarly provide access to both conductive layers from a same side of the needle sensor.

FIGS. 8B and 8C show in greater detail two potential stepped arrangements in the context of a needle sensor that includes three conductive layers instead of two. It is first noted that even a needle sensor that includes three electrodes may not require a stepped arrangement, and the layers can all be similarly sized, similar to the arrangement shown in FIG. 8A. In such an embodiment, access to the middle conductive layer can still be provided via electrical connectivity around the exposed end or side walls of the middle conductive layer. However, the arrangements in FIGS. 8B and 8C show options that would likely provide an easier and potentially more robust connection, particularly to the middle conductive layer.

In the arrangement shown in FIG. 8B, a rear end of a needle sensor with three conductive layers 181′, 182′, 183′ is shown. The three conductive layers 181′, 182′, 183′, are respectively insulated from one another by insulating layers 184′, 185′. A first conductive layer 181 has an exposed face on one side of the needle sensor, and a second conductive layer 182 has an exposed face on an opposite side of the needle sensor. Meanwhile, the middle conductive layer 183′ may include an extended region that forms its electrical contact, to provide a more significant end region for easier and/or more robust connectivity. The extended end region of the middle conductive layer 183′ may be formed, for example, by fabricating it with an extended footprint during the layering process, or for example, by removing respective material from conductive layers 181′, 182′ and insulating layers 184′, 185′, to more significantly expose the end region of conductive layer 183′. In this arrangement, the port or other electrical contacts in the CGM may still be configured to communicate with both sides of the needle sensor, to more readily access both the first and second conductive layers 181′, 182′.

In a still further alternative arrangement shown in FIG. 8C, a rear end of a needle sensor with three conductive layers 181″, 182″, 183″ is shown. The three conductive layers 181″, 182″, 183″, are respectively insulated from one another by insulating layers 184″, 185″. In FIG. 8C, the electrical contacts formed by the conductive layers 181″, 182″, 183″ are arranged in a stair or step-like manner, so that the electrical contact surfaces of all of the conductive layers of the needle sensor face a same way and are more easily accessible from a single side or orientation of the needle sensor. Here, a bottom conductive layer 181″ (as illustrated) may extend out farther from the rear end of the needle sensor than the middle conductive layer 183″ and the insulating layer 184″, which in turn may extend out farther from the rear end of the needle sensor than the top conductive layer 182″ (as illustrated) and the insulating layer 185″. Using such an arrangement, all of the conductive layers 181″, 182″ 183″ can more easily be accessed by external circuitry from the same top face of the needle sensor (as illustrated). While such an arrangement may require more space to implement, it has the advantage of allowing external electrical contact from the same side for all of the conductive layers.

As with the previous embodiments, needle sensors with three conductive layers can also be arranged with various different modifications without departing from the scope of the invention. For example, the stepped arrangements are only one design option, and in alternative embodiments, only select portions of each layer, for example, the same or different corners of the conductive layers, may be arranged differently from the other respective layers. In addition, since for example, the embodiment in FIG. 8C provide electrical contacts all facing a first side or face of the needle sensor, the outer insulating layer may be extended to cover more or all of the opposite side of the needle sensor, the side walls, and/or the end face of the bottom conductive layer (as illustrated), to provide better insulation for portions of the needle sensor where electrical connectivity is not needed or desired.

Other modifications can also be made without departing from the spirit of the invention. For example, in some embodiments, the needle sensor may be modified to include four or more electrodes or conductive layers, where in such embodiments, both the sensor tip and electrical contact arrangements can be correspondingly modified to accommodate any such additional electrodes or conductive layers. In addition, different features from the different embodiments can also be combined to provide still further embodiments not specifically described herein.

According to embodiments of the invention, a needle sensor can be easily and reliably manufactured, which provides a single part that can serve the dual purpose of providing a sharp tip for penetrating biological tissue as well as providing electrically active sensing electrodes into a region of interest, without the need to introduce a separate fiber or other component to do so. In some embodiments, one or more of the conductive layers may be made of or include, for example, a biocompatible metal or other rigid conductive material, to facilitate strengthening of the needle sensor to serve its dual purposes. Similarly, one or more of the insulating layers may be made of or include, for example, a rigid biocompatible insulating material to impart additional structural strength to the needle sensor. Utilizing such needle sensors according to embodiments of the invention will simplify the CGM application process for end users, reduce pain, and increase sensing accuracy, among various other potential benefits.

While the present invention has been described in connection with certain exemplary embodiments, it is to be understood that the invention is not limited to the disclosed embodiments, but is instead intended to cover various modifications and equivalent arrangements included within the spirit and scope of the appended claims, and equivalents thereof.

What is claimed is:

1. A sensor for facilitating analysis of biological specimens in vivo, comprising:
a base having a first end and a second end;
a rigid needle that extends away from the first end for piercing skin, the needle having a tip comprising at least a first conductive layer forming a first electrode, a second conductive layer forming a second electrode, and a first insulating layer between and electrically insulating the first and second conductive layers from one another; and
at least first and second electrical contacts at or near the second end of the base, wherein the first electrical contact is in electrical communication with the first electrode, the second electrical contact is in electrical communication with the second electrode, and the first and second electrical contacts are electrically insulated from one another.

2. The sensor of claim 1, wherein the sensor has a substantially planar first face and an opposite substantially planar second face that extend parallel to one another from the first end of the base towards the second end of the base.

3. The sensor of claim 2, wherein the first electrode is exposed on a side of the first face and the second electrode is exposed on a side of the second face.

4. The sensor of claim 2, wherein the first electrical contact is exposed on a side of the first face and the second electrode is exposed on a side of the second face.

5. The sensor of claim 2, wherein both the first and second electrical contacts are exposed on a side of the first face.

6. The sensor of claim 1, further comprising an external insulating layer that exposes the electrodes and the electrical contacts while covering other parts of the sensor.

7. The sensor of claim 6, wherein the external insulating layer covers a shaft of the needle that extends between the tip of the needle and the first end of the base.

8. The sensor of claim 1, wherein the tip of the needle further comprises a third conductive layer forming a third electrode between the first and second electrodes, and a second insulating layer, with the first and second insulating layers respectively insulating the third conductive layer from the first and second conductive layers, and a third electrical contact at or near the second end of the base that is in electrical communication with the third electrode and that is insulated from the first and second electrical contacts.

9. The sensor of claim 8, wherein the sensor has a substantially planar first face and an opposite substantially planar second face that extend parallel to one another from the first end of the base towards the second end of the base, and wherein at least two of the electrical contacts are exposed on a side of the first face.

10. The sensor of claim 1, wherein at least one of the first or second conductive layers comprises a biocompatible metal.

11. A wearable device for analyzing biological specimens in vivo, comprising:
a housing;
a holding element for holding the housing against a patient's skin; and
a needle configured to extend away from the housing to pierce the skin, wherein a tip of the needle that pierces the skin is configured to remain under the skin and comprises at least first and second electrodes that are electrically insulated from one another.

12. The wearable device of claim 11, wherein the holding element comprises an adhesive applicable against the skin.

13. The wearable device of claim 11, further comprising:
circuitry configured to electrically connect the needle to a controller;
a transmitter/receiver for transmitting data outside of the wearable device; and
a battery for supplying power to the wearable device.

14. The wearable device of claim 11, wherein when the wearable device is worn against the skin, the tip of the needle is configured to be held under the skin until the entire wearable device is removed, to provide continuous in vivo analysis of the patient.

15. A method of forming a sensor that facilitates analysis of biological specimens, the method comprising:
depositing a first insulating layer on a first conductive layer;
depositing a second conductive layer on the first insulating layer, such that the first and second conductive layers are electrically insulated from one another;
shaping the first and second conductive layers and the first insulating layer into a shape that comprises a base having a first end and a second end, a rigid needle that extends away from the first end for piercing skin, wherein the first and second conductive layers are exposed and respectively form first and second electrodes at a tip of the needle, and at least a first electrical contact in electrical communication with the first electrode and a second electrical contact in electrical communication with the second electrode at or near the second end of the base.

16. The method of claim 15, further comprising depositing the first conductive layer.

17. The method of claim 15, wherein at least one of the first or second conductive layers or the first insulating layer comprises a rigid material.

18. The method of claim 15, further comprising depositing a second insulating layer on the second conductive layer, and depositing a third conductive layer on the second insulating layer, such that the third conductive layer is electrically insulated from the first and second conductive layers.

19. The method of claim 15, wherein exposed portions of the first and second electrical contacts have at least one of a different size or a different shape from one another.

20. The method of claim 15, further comprising forming an external insulating layer that exposes the electrodes and the electrical contacts while covering other parts of the sensor.
